(19) United States
(12) Patent Application Publication
(10) Pub. No.: US 2011/0076700 A1
(43) Pub. Date: Mar. 31, 2011

(54) ANTI-CRP ANTIBODY AND UTILIZATION OF THE SAME

(75) Inventors: Hideki Kohno, Tokyo (JP); Maho Kikuchi, Tokyo (JP); Tomoe Komoriya, Tokyo (JP)

(73) Assignee: Nihon University, Tokyo (JP)

(21) Appl. No.: 12/920,192

(22) PCT Filed: Aug. 29, 2008
(86) PCT No.: PCT/JP08/02376

§ 371 Date: Aug. 30, 2010

(30) Foreign Application Priority Data
Feb. 29, 2008 (JP) 2008-051165

(51) Int. Cl. G01N 33/53; C07K 16/18; C12N 5/00; G01N 33/566

(52) U.S. Cl. 435/7.92; 530/387.9; 530/387.3; 435/346; 436/501

(57) ABSTRACT

It is intended to provide means capable of specifically recognizing CRP in a test sample and assaying it with high sensitivity. The present invention provides an anti-CRP antibody which reacts with a C-reactive protein (hereinafter referred to as CRP) and recognizes an epitope located at residues 147 to 172 in a CRP amino acid sequence represented by SEQ ID NO: 1.

### TECHNICAL FIELD

The present invention relates to an anti-CRP antibody and utilization of the same. More specifically, the present invention relates to a method for assaying CRP with specificity and high sensitivity, including identifying a reaction site on CRP recognized by an anti-CRP antibody using an epitope analysis method and conducting immunoassay using an antibody prepared based on the reaction site.

### BACKGROUND ART

A C-reactive protein (CRP) is a serum protein that exhibits precipitation reaction with C-polysaccharide of Diplococcus pneumoniae and is usually present in trace amounts (580 ng/ml on average) in normal human serum. CRP is characterized by being rapidly increased in blood in response to inflammatory disease or tissue degeneration/necrosis and rapidly decreased with convalesce from the disease. Thus, the assay of CRP concentrations in blood is clinically used in a wide range of diagnoses of inflammations (e.g., rheumatoid arthritis, bacterial infection, viral hepatitis, pneumonia, and urinary tract infection) or tissue-destroying disease (see Non-Patent Document 1).

Conventional CRP assay involves measuring drastic increase from the normal concentration occurring during acute inflammation and therefore less required an assay method with high sensitivity. However, CRP has been confirmed in recent years to be useful as a marker for predicting ischemic cardiac disease (myocardial infarction), neonatal infection, or the like and has required precision and high sensitivity for assay thereof. Moreover, a link between CRP and various diseases such as periodontal disease has also been pointed out, and the value of assay of CRP in trace amounts (low concentration) is thus of great clinical significance (see Non-Patent Document 2).

On the other hand, an anti-CRP antibody used in CRP assay is prepared mainly with natural proteins as antigens. Therefore, this method has many problems that, for example, nonspecific reaction tends to occur; and when purified proteins are obtained from human serum, there are ethical concerns and lot-to-lot variations of products.

Thus, a method for preparing an antibody using recombinant human CRP (rCRP) has also been proposed and however, is less than sufficient for binding specificity.
[Non-Patent Document 1] Fukuoka Y. et al., Clinical Immunology, Ishiyaku Pub., Inc., 1997
[Non-Patent Document 2] Takahashi H., “Usefulness of Highly Sensitive CRP Assay Method in Disease Diagnosis,” Japanese Journal of Clinical Pathology, 2002, vol. 50, p. 30-39

### DISCLOSURE OF THE INVENTION

### Problems to be Solved by the Invention

An object of the present invention is to provide means capable of specifically recognizing CRP in a test sample and assaying it with high sensitivity.

### Means for Solving the Problems

A region on CRP recognized and bound by an anti-CRP antibody has been unknown so far, and an anti-CRP antibody which has been shown to recognize a specific site in CRP has not been obtained. Thus, the present inventors have analyzed a reaction site on CRP recognized by an anti-CRP antibody using an epitope analysis method and consequently found an antibody having antigen specificity for residues 147 to 172 in a CRP amino acid sequence. The present inventors have further found that immunoassay using an antibody recognizing the region can assay CRP in a test sample with exceedingly high specificity and high sensitivity. Based on the findings, the present invention has been completed.

Specifically, the present invention provides an anti-CRP antibody which reacts with CRP and recognizes an epitope located at residues 147 to 172 in a CRP amino acid sequence represented by SEQ ID NO: 1.

Moreover, the present invention provides a hybridoma CRP8 producing the antibody.

Moreover, the present invention provides a CRP assay reagent containing the antibody.

Furthermore, the present invention provides a CRP assay method including bringing the antibody into contact with a test sample, followed by immunoassay.

### EFFECT OF THE INVENTION

According to the present invention, CRP in a test sample can be detected with exceedingly high sensitivity due to specific reaction. Therefore, this approach can capture even very minor inflammations in the body, such as local inflammation or lesions at small sites and is thus exceedingly useful in the clinical assay, for example, the detection of various diseases and the determination of severity, prognosis, or therapeutic effect. Moreover, use of recombinants in antibody preparation can be expected to bring about cost reduction in CRP assay reagent.

### BRIEF DESCRIPTION OF THE DRAWINGS

FIG. 1 is a diagram showing CRP genes amplified by PCR;

FIG. 2 is a diagram showing results of the reactivity of an anti-CRP monoclonal antibody-sensitized latex reagent; and

FIG. 3 is a diagram showing results of assaying a CRP concentration in a liver disease specimen.

### BEST MODE FOR CARRYING OUT THE INVENTION

Human CRP is a protein that consists of 5 subunits and has a molecular weight of 105,000 Da. Its amino acid sequence is known in the art and described in, for example, J. Biol. Chem., 260, 13377-13383, 1985.

An anti-CRP antibody of the present invention has reactivity to human CRP and recognizes an epitope located at residues 147 to 172 in a CRP amino acid sequence represented by SEQ ID NO: 1. The antibody encompasses both monoclonal and polyclonal antibodies. Particularly preferable examples thereof can include a monoclonal antibody that is produced by a hybridoma CRP8 deposited under Accession No. FERM ABP-11001 and recognizes an epitope located at residues 147 to 172 in a CRP amino acid sequence represented by SEQ ID NO: 1.

Such an anti-CRP antibody recognizing the particular region as an epitope can be obtained by a method known in the art and can be obtained, for example, by using a peptide containing the amino acid sequence of the region as an immunizing antigen. Moreover, the antibody can also be obtained by determining an epitope recognized by the obtained antibody after antibody preparation according to a standard method and selecting antibodies recognizing the epitope of interest. In this context, the epitope for the antibody can be determined by ELISA, western blot, or the like.

In the present invention, natural human CRP, recombinant CRP (rCRP), a peptide having the amino acid sequence represented by SEQ ID NO: 1, or the like can be used as an immunizing antigen. Particularly, the peptide having the amino acid sequence represented by SEQ ID NO: 1 is preferably used for obtaining antibodies with high-titer and highly specificity. As described above, antibody production using natural human CRP is a well known method, however, has many problems that, for example, nonspecific reaction often occurs in immunoassay; and when purified proteins are obtained from human serum, there are ethical concerns and lot-to-lot variations of products. By contrast, the method using the peptide having the amino acid sequence represented by SEQ ID NO: 1 is free from the problem associated with starting materials and is exceedingly useful because the peptide is readily available from facilities well equipped for protein expression or peptide synthesis and is also highly specific in assay precision or accuracy.

The peptide having the amino acid sequence represented by SEQ ID NO: 1 can be produced by a genetic engineering approach, a peptide synthesis method known in the art, or appropriate peptidase cleavage of CRP. The peptide synthesis may be performed by, for example, any of solid-phase and liquid-phase synthesis methods.

Among them, the genetic engineering approach is preferable. The recombinant peptide can be prepared by a method known in the art, for example, by: cloning, into expression vectors, a polynucleotide encoding the peptide containing residues 147 to 172 in the CRP amino acid sequence (also including peptides having substantially the same activity thereas); subsequently transforming appropriate host cells such as E. coli with the obtained recombinant plasmids; culturing the obtained transformants under conditions capable of causing expression; and separating and purifying the desired peptide from the cultures. Whether or not to obtain the protein of interest can be confirmed by SDS-polyacrylamide gel electrophoresis or the like.

In this context, the polynucleotide encoding residues 147 to 172 in the CRP amino acid sequence can be prepared by a method routinely used in the genetic engineering field, for example, a method including amplifying the gene of interest by PCR using appropriate primers prepared based on the information of the CRP amino acid sequence.

When the peptide is used as an immunizing antigen, the peptide is preferably bonded, for immunization, to a carrier such as: mammal-derived proteins such as albumin and globulin; proteins such as keyhole limpet hemocyanin; microorganisms such as inactivated tubercle bacillus; and polyamino acids such as polylysine and polyasparagine.

The monoclonal antibody of the present invention is produced, according to a method known in the art for monoclonal antibodies, by a hybridoma obtained by fusing antibody-producing cells of antigen-immunized mammals with mammalian myeloma cells.

Specifically, mammals are first immunized with sensitizing antigens by intraperitoneal, subcutaneous, intravascular, intramuscular, or intrasplenic injection or the like or by oral administration. In this context, the mammals used in immunization are not particularly limited and are preferably selected in consideration of compatibility with myeloma cells used in cell fusion in the subsequent operation. Specific examples thereof include mice and rats. Specifically, the sensitizing antigens are diluted or suspended into appropriate amounts with PBS (phosphate-buffered saline), saline, or the like and mixed, if desired, with an appropriate amount of a usual adjuvant, for example, a Freund's complete adjuvant. After emulsification, the resultant is administered to mammals several times at 4- to 21-day intervals.

Next, splenic cells collected from the immunized animals are fused with myeloma cells of mammals such as mice. The myeloma cells are preferably those having an appropriate marker of hypoxanthine-guanine-phosphoribosyltransferase deficiency (HGPRT⁻), thymidine kinase deficiency (TK⁻) or the like. Specific examples thereof include mouse P3/NS1/1-Aq4-1. The fusion can be performed according to an approach known in the art. Moreover, polyethylene glycol (PEG), Hemagglutinating virus of Japan (HVJ), or the like can be used as a fusion promoter. The splenic cells and the myeloma cells are preferably mixed at a ratio of 1:1 to 10:1. According to circumstances, the cell fusion can also be performed by electrofusion or the like.

After the cell fusion, the cells can be cultured in a usual medium for selection to selectively obtain hybridomas. When the colony becomes sufficiently large, a strain producing the antibody of interest is searched for and prepared as single clones.

The hybridomas can be screened for by culturing the hybridomas in, for example, a microplate, and assaying the reactivity of the grown hybridomas in culture supernatant in wells to the sensitizing antigen used in the mammal immunization by using a method generally used in antibody detection, for example, enzyme immunoassay. Examples of the enzyme immunoassay include ELISA and RIA. In this context, hybridomas producing the antibody of the present invention can be selected efficiently by evaluating the reactivity not only to human CRP but to the amino acid sequence of residues 147 to 172.

The preparation of the selected hybridomas as single clones can be performed by, for example, a limiting dilution or soft agar method. In this procedure, mouse thymocytes, peritoneal macrophages, or a known additive having the same effect thereas is preferably used as a feeder.

To produce the antibody of the present invention using the obtained monoclonal hybridomas, the hybridomas may be cultured in an appropriate medium or in the abdominal cavities of mice or the like. The medium used here is not particularly limited as long as it is a medium suitable for hybridoma culture. For example, an RPMI 1640 medium containing fetal bovine serum, L-glutamine, L-pyruvic acid, and antibiotics (penicillin G and streptomycin) is preferable. The culture is preferably performed, for example, under conditions involving a 5% CO₂ concentration and 37° C. for approximately 2 to 4 days, on the hybridomas added at a concentration of 10⁴ to 10⁵ individuals/ml to the medium. The supernatant obtained by this culture can be subjected to centrifugation or the like to obtain the antibody of the present invention. On the other hand, the culture in the abdominal cavities may be performed by intraperitoneally administering the hybridomas to mice and collecting the ascites.

The monoclonal antibody of the present invention in the culture supernatant may be used directly or may be used after purification by using, for example, fractionation by ammonium sulfate precipitation, ion-exchange chromatography, a protein A-bound carrier, or an anti-IgG antibody column.

The polyclonal antibody of the present invention is prepared according to a method known in the art for polyclonal antibodies. Specifically, the polyclonal antibody can be obtained by: immunizing the same mammals as those exemplified above with the peptide containing residues 147 to 172 in the CRP amino acid sequence as an antigen; and collecting serum containing produced antibodies having reactivity to human CRP. The polyclonal antibody can be used directly or may be used after purification in the same way as above.

The specificity of the anti-CRP antibody of the present invention can be confirmed by, for example, western blot or ELISA.

The purity of the antibody is not particularly limited, and, for example, both globulin and affinity-purified fractions may be used. Moreover, the anti-CRP antibody of the present invention is not limited to the whole antibody molecule and may be an antibody fragment or a modified form thereof as long as it binds to CRP. A divalent or monovalent antibody is also included in the antibody of the present invention. Examples of the antibody fragment include Fab, F(ab′)₂, Fv, Fab/c having one Fab and complete Fc, and single chain Fv (scFv) having H or L chain Fvs connected via an appropriate linker.

The anti-CRP antibody of the present invention obtained thus is useful for the immunoassay of human CRP in a test sample. In this context, the test sample is not particularly limited as long as it is a sample that is likely to contain CRP. Specific examples thereof can include blood, interstitial fluid, plasma, extravascular fluid, cerebrospinal fluid, synovial fluid, pleural fluid, serum, lymph, saliva, and urine. Moreover, a sample obtained from a test sample such as a culture solution of cells collected from the living body is also included in the test sample of the present invention.

The immunoassay is not particularly limited, and, for example, Ouchterlony method, single immunodiffusion, immunonephelometry, enzyme immunoassay, latex immunoassay, radioimmunoassay, and fluoroimmunoassay can be used. Of them, a latex agglutination method which utilizes agglutination reaction typified by ELISA and LPIA is preferable. In this context, the assay encompasses quantitative and non-quantitative assays. Examples of the non-quantitative assay include a qualitative method including determining the degree of formed agglutinates by visual observation to be negative (−) or positive (+). Examples of the quantitative assay can include determining CRP concentration or CRP amount.

The anti-CRP antibody of the present invention can be labeled with a labeling material, if necessary, for use. Examples of the labeling material include: enzymes such as peroxidase, alkaline phosphatase, β-D-galactosidase, and glucose oxidase; radioisotopes such as ³²P, ¹⁴C, ¹²⁵I, ³H, and ¹³¹I; fluorescent materials such as fluorescein isothiocyanate and rhodamine; and chemical substances such as biotin, avidin, and digoxigenin.

When the labeling material is an enzyme, a substrate and, if necessary, a coloring agent are used for measuring the activity. When peroxidase is used as the enzyme, hydrogen peroxide is used as a substrate and o-phenylenediamine, 3,3′,5,5′-tetramethylbenzidine, 2,2′-azino-di-[3-ethylbenzthiazoline sulfonic acid]ammonium salt, or the like is used as a coloring agent; when alkaline phosphatase is used as the enzyme, p-nitrophenyl phosphate, 3-(4-methoxyspiro{1,2-dioxetane-3,2′-tricyclo-[3.3.1.1^{3,7}]decan}-4-yl)phenyl phosphate (AMPPD), or the like is used as a substrate; when β-D-galactosidase is used as the enzyme, β-D-galactopyranoside, 4-methylumbelliferyl-β-D-galactopyranoside, or the like is used as a substrate; and when glucose oxidase is used as the enzyme, β-D-glucose as a substrate and peroxidase as a coloring agent can be used in the presence of peroxidase.

The enzyme-labeled antibody used can be prepared by a method known in the art, for example, by labeling, with the enzyme, either an unfragmented immunoglobulin molecule directly or F(ab′)₂ or Fab′ obtained by subjecting an antibody to limited-degradation with an appropriate protease according to the need, according to the method of Nakane et al. (Nakane P. K et al., J. Histochem Cytochem, 22, 1084-1089, 1974) or the method of Ishikawa et al. (maleimide method: “Enzyme Immunoassay 3rd ed.”, Igaku-Shoin Ltd.) or the like.

Moreover, the anti-CRP antibody of the present invention can also be immobilized on an insoluble carrier and used as an immobilized enzyme. The insoluble carrier is preferably various synthetic polymers such as polystyrene, polyethylene, and polypropylene, glass, silicon, insoluble polysaccharide (cross-linked dextran or polysaccharide), or the like. These carriers can be used, for example, in a spherical, rod-like, or fine particle shape or in a test tube or microplate form. The insolubilized antibody can be preferably prepared, for the spherical, rod-like, test tube, or microplate form and for the fine particle form, at antibody concentrations of 1 to 10 μg/ml and 1 to 10 mg/ml, respectively, in a neutral to alkaline buffer solution with pH 7 to 10, such as a phosphate buffer, glycine buffer, carbonate buffer, or tris buffer, at room temperature or 4° C. for 1 hour to 72 hours.

The binding between the anti-CRP antibody and CRP is usually performed in a buffer. The buffer is not particularly limited as long as it is a solution capable of causing antigen-antibody reaction. A phosphate buffer, glycine buffer, tris salt buffer, Good buffer, or the like is preferable. The reaction pH is preferably in the range of pH 7 to 9. Moreover, water-soluble polymers such as polyethylene glycol, polyvinylpyrrolidone, and pullulan; stabilizers such as bovine serum albumin and sucrose; preservatives such as sodium azide; and additives such as sodium chloride may be added appropriately to the reaction solution for the purpose of sensitivity improvement, reaction promotion, or stabilization.

The CRP assay reagent of the present invention may contain, in addition to the anti-CRP antibody, bovine serum albumin, sucrose, or the like appropriately dissolved in terms of prevention of nonspecific reaction and storage stability and sodium chloride or the like dissolved for salt concentration adjustment.

Furthermore, the CRP assay reagent of the present invention may be a one-component reagent containing the anti-CRP antibody dispersed or dissolved therein or may be used as a two-component or three-component reagent. Moreover, the reagent encompasses a kit. The kit may appropriately contain a blocking solution, a reaction solution, a reagent for sample treatment, and the like.

The concentration of the antibody against CRP in the CRP assay reagent of the present invention is not particularly limited as long as it is a concentration at which CRP in a test sample can be assayed. The concentration is preferably set to 50 to 400 μg/mL, particularly preferably 100 to 200 μg/mL. At a concentration lower than 50 μg/mL, accurate quantification is hardly achieved in a low concentration region due to low sensitivity. On the other hand, at a concentration exceeding 400 μg/mL, nonspecific reaction tends to occur.

A conventional CRP quantification method using LPIA has sensitivity of approximately 500 ng/mL and cannot capture local inflammation or lesions at small sites. However, the present invention has sensitivity as exceedingly high as 3 to 5 ng/mL and can capture even very minor inflammation in the body. Thus, the CRP assay reagent of the present invention is preferably used in the diagnosis, therapeutic follow-up, or the like of various infections, inflammatory diseases, and tissue-destroying diseases.

Examples of such diseases include inflammations (e.g., rheumatism, bacterial infection, viral hepatitis, pneumonia, macular degeneration, and urinary tract infection), tissue-destroying disease, ischemic cardiac disease, neonatal infection, periodontal disease, and aggravated inflammation in the tonsillitis palatina.

### EXAMPLES

Hereinafter, the present invention will be described specifically with reference to Examples. However, the present invention is not limited to them by any means.

### Example 1

### Preparation of Anti-CRP Antibody

[Preparation of rCRP]

### (1) Preparation of CRP Gene

DNA was extracted from leukocyte components in human blood. To the whole blood, a 3-fold volume of an EDTA solution was added for the DNA extraction. This DNA was used as a template to PCR amplify 6 types of gene fragments using 7 types of primers constructed based on the CRP gene sequence (J. Biol. Chem., 260, 13377-13383, 1985). The primers used were an MK01 (SEQ ID NO: 2), MK02 (SEQ ID NO: 3), MK03 (SEQ ID NO: 4), MK04 (SEQ ID NO: 5), MK05 (SEQ ID NO: 6), or MK06 (SEQ ID NO: 7) forward primer and a reverse primer (SEQ ID NO: 8) described in Table 1.

TABLE 1
Forward & Reverse primer for recombinant CRP

| Primer | Sequence | Tm(° C.) | Length (bp) |
|---|---|---|---|
| MK01 | 5′-CACCCAGACAGACATGTCGAGGAAGGTT-3′ | 75.0 | 618 |
| MK02 | 5′-CACCATGTCGAGGAAGGCTTTTG-3′ | 70.5 | 612 |
| MK03 | 5′-CACCTCGTATGCCACCAAGAGACA-3′ | 71.1 | 465 |
| MK04 | 5′-CACCAGGGTGAGGAAGAGTCTGAAG-3′ | 70.1 | 276 |
| MK05 | 5.-CACCGAAGGAAGCCAGTCCCT-3′ | 70.6 | 183 |
| MK06 | 5′-CACCACCATCTATCTTGGCGGG-3′ | 71.3 | 105 |
| Reverse | 5′-AAACCCCAGCTGTGGCCCTGA-3′ | 73.9 | — |

* Forward primers (MK01 to MK06) are those added CACC
necessary for genetic recombination added before the CRP
sequence

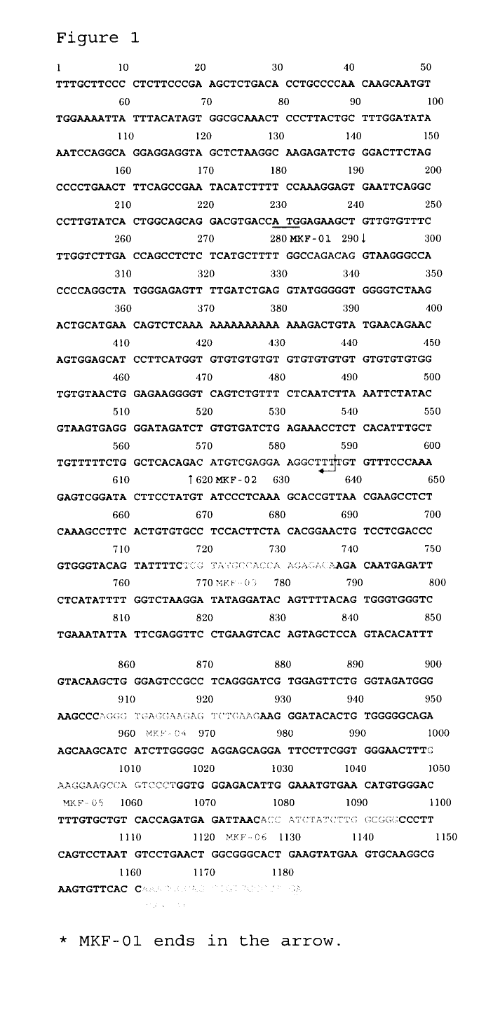

The 6 types of DNA fragments (FIG. 1) amplified by PCR were purified using crystal violet gel according to a standard method.

(2) Next, genetic recombination was performed using E. coli as host cells. Each DNA fragment obtained above was subcloned into pET-100/D-TOPO vectors (Invitrogen Corp.), with which an E. coli host Top10 was then transfected on ice. The whole amount thereof was seeded onto an LB Ampicillin plate and incubated at 37° C. for 15 hours to form single colonies. To examine the presence of the plasmid of interest in the formed colonies, colony PCR was performed using primers for the insert gene. Only the colony whose band could be confirmed was subcultured in an LB Ampicillin plate and cultured with stirring (200 rpm) at 37° C. for 15 hours.
(3) The plasmids obtained by the procedures were used in the transfection of an E. coli host BL21. The whole amount thereof was seeded onto an LB Ampicillin plate and incubated (200 rpm) at 37° C. for 15 hours to form single colonies. The colonies were collected and subcultured in an LB medium containing ampicillin. At the point in time of OD₆₀₀=0.5, the culture solution was divided into two portions. IPTG was added at a final concentration of 1.0 mM only to one of the culture solutions to induce the expression of partial recombinant human CRP. Then, the culture solution was hourly collected (0 to 5 hours). The collected culture solution was centrifuged at 16,000×g for 1 hour, and the obtained pellet was then frozen.

### (4) Confirmation of Expressed Protein by SDS-PAGE

The bacterial cell pellet of E. coli collected above was dissolved in a lysis buffer for protein dissolution, and the bacterial cells were disrupted by freezing and thawing using liquid nitrogen and centrifuged. Proteins in the obtained supernatant and pellet were confirmed by SDS-PAGE electrophoresis.

As a result, bands could be detected for the 6 types of expressed proteins. Bands of the expressed proteins have the molecular weight of a His tag protein (4774 Da) bound with 26.7 kDa of MK01, 26.3 kDa of MK02, 19.9 kDa of MK03, 11.7 kDa of MK04, 6.8 kDa of MK05, or 4.0 kDa of MK06, and these molecular weights were all consistent with the molecular weights of the bands.

### [Preparation of Antibody]

Balb/C mice (CRL) were immunized with each rCRP prepared above. For initial immunization, the immunizing protein was prepared at 100 μg/mouse and emulsified using FCA (Freund's complete adjuvant (H37 Ra), Difco (3113-60), and Becton, Dickinson and Company (cat #231131)), and this emulsion was subcutaneously administered to the mice. Two weeks later, the immunizing protein was further prepared at 50 μg/mouse and emulsified using FIA (Freund's incomplete adjuvant, Difco (0639-60), and Becton, Dickinson and Company (cat #263910)), and this emulsion was subcutaneously administered to the mice. Then, booster immunization was performed a total of 5 times at 1-week intervals. For the final immunization, the antigen was diluted with PBS into 50 μg/mouse and administered to the tail veins. After confirmation that the antibody titer in serum to CRP reached a level of saturation by ELISA using an immunoplate, the mouse splenic cells were mixed with mouse myeloma cells P3U1 to perform cell fusion using PEG1500 (Roche Diagnostics, cat #783 641). The cells were seeded onto a 96-well culture plate. After selection in an HAT medium on the next day, the culture supernatant was subjected to screening by ELISA.

Positive clones were prepared as single clones by a limiting dilution method and then cultured for expansion, and the culture supernatant was collected. The screening was performed using ELISA and binding activity to CRP as an index, and anti-CRP monoclonal antibodies having strong binding ability were obtained.

The antibodies were purified using Hi Trap Protein G HP (Amersham Biosciences Corp., CAT #17-0404-01). The hybridoma culture supernatant was directly charged to the column and washed with a binding buffer (20 mM sodium phosphate (pH 7.0)), followed by elution with an elution buffer (0.1 M glycine-HCl (pH 2.7)). The elution was performed in a tube supplemented with a neutralizing buffer (1 M Tris-HCl (pH 9.0)), and the eluate was immediately neutralized. The antibody fractions were pooled and then dialyzed one whole day and night by using 0.05% Tween 20/PBS, and the buffer was replaced. The purified antibodies were stored at 4° C. after addition of NaN₃ achieving a 0.02% concentration.

Of the obtained hybridomas, the hybridoma producing a monoclonal antibody Lot. 050921 was designated as CRP8 and deposited (Accession No: FERM ABP-11001) on Aug. 28, 2008 with International Patent Organism Depositary, National Institute of Advanced Industrial Science and Technology (address: Tsukuba Central 6, 1-1-1 Higashi, Tsukuba, Ibaraki).

The isotyping of the anti-CRP monoclonal antibodies was performed using ImmunoPure Monoclonal Antibody Isotyping Kit II (PIERCE CAT #37502) by a method following the manual included therein. As a result of isotyping, all the antibodies were of IgG1 type.

Of the obtained antibodies, the anti-CRP monoclonal antibody Lots. 050921, 030700, and CP80105 were used in the following experiments.

### Example 2

### Confirmation of Site Recognized by Antibody by Sandwich ELISA

The epitope analysis of the anti-CRP monoclonal antibodies prepared in Example 1 was conducted using the expressed proteins (MK01 to MK06).

A capture antibody (anti-CRP IgG rabbit serum) diluted to 10 μg/ml with a sodium carbonate buffer was added at a concentration of 50 μL/well to the 96-well plate and left standing at 37° C. for 1 hour. The antibody solution was removed, and a 6% blocking buffer was added thereto at a concentration of 100 μL/well and left standing at 37° C. for 1 hour. The plate was washed with PBS-T. Then, the expressed protein (antigen) diluted with a lysis buffer was added thereto at a concentration of 50 μL/well and left standing at 37° C. for 1 hour. The plate was washed with PBS-T. Then, a primary antibody (anti-CRP monoclonal antibody) diluted to 0.5 mg/ml with PBS was added thereto and left standing at 37° C. for 1 hour. The plate was washed with PBS-T. Then, a secondary antibody (anti-mouse IgG goat serum antibody labeled with HRP) diluted to 0.1 μg/ml with PBS was added thereto and left standing at 37° C. for 1 hour. The plate was washed with PBS-T. Then, a coloring solution was added thereto at a concentration of 50 μL/well, and the plate was left for 20 minutes under shading. After confirmation of coloring, 50 μL of a reaction stop solution (1 N sulfuric acid) was added to the coloring solution, and the absorbance was measured at a wavelength of 492 nm using a microplate reader.

As a result, the antibody of Lot. 050921 did not react with MK06 as shown in Table 2. Therefore, the epitope was demonstrated to be located at residues 147 to 172 in the amino acid sequence represented by SEQ ID NO: 1, i.e., the CRP amino acid sequence. Moreover, the epitopes for the antibodies of Lot. 030700 and Lot. CP80105 were demonstrated to be located at residues 173 to 206 in the amino acid sequence represented by SEQ ID NO: 1.

TABLE 2
Result of ELISA with Cell line anti CRP IgG monoclonal

| | | Reactivity with Recombinant CRP | | | | | |
|---|---|---|---|---|---|---|---|
| | | ELISA | | | | | |
| MoAb | Isotype | MK01 | MK02 | MK03 | MK04 | MK05 | MK06 |
| MoAb Lot.050921 | IgG1κ | + | + | + | + | + | − |
| MoAb Lot.030700 | IgG1κ | + | + | + | + | + | + |
| MoAb Lot.CP80105 | IgG1κ | + | + | + | + | + | + |

### Example 3

### Confirmation of Specificity of Anti-CRP Antibody by Western Blotting

The expressed proteins obtained above were electrophoresed using a 12.5% polyacrylamide gel and then transferred to a PVDF filter, which was then blocked for 1 hour. The filter was washed with PBS-T and then reacted for 1 hour with the anti-CRP monoclonal antibody diluted to 1.0 μg/ml with PBS-T. The filter was washed with PBS-T and then reacted for 1 hour with an anti-mouse IgG goat serum antibody labeled with HRP diluted to 0.2 μg/ml with PBS-T. The filter was washed with PBS-T and then photosensitized with a chemiluminescence detecting reagent.

As a result, as is evident from Table 3, the antibody of Lot. 050921 did not react with MK06. Therefore, the epitope was demonstrated to be located at residues 147 to 172 in the amino acid sequence represented by SEQ ID NO: 1, i.e., the CRP amino acid sequence. Moreover, the epitopes for the antibodies of Lot. 030700 and Lot. CP80105 were demonstrated to be located at residues 173 to 206 in the amino acid sequence represented by SEQ ID NO: 1.

TABLE 3
Result of Western Blot with cell line anti CRP IgG monoclonal

| | | Reactivity with Recombinant CRP | | | | | |
|---|---|---|---|---|---|---|---|
| | | Western Blot | | | | | |
| MoAb | Isotype | MK01 | MK02 | MK03 | MK04 | MK05 | MK06 |
| MoAb Lot.050921 | IgG1κ | + | + | + | + | + | − |
| MoAb Lot.030700 | IgG1κ | + | + | + | + | + | + |
| MoAb Lot.CP80105 | IgG1κ | + | + | + | + | + | + |

### Test Example 1

### Evaluation of Anti-CRP Antibody

### (1) Method for Preparing Latex Reagent

2.0 mL of a WSC solution with a concentration of 20 mg/mL and 0.23 mL of an NHS solution with a concentration of 50 mg/mL were added in this order with stirring to a 1% suspension of carboxyl group-modified latex particles (“Immutex” manufactured by JSR Corp.) to activate the carboxyl group on the latex surface. After the activation, the mixture was centrifuged (16000 rpm, 4° C., 20 min) and divided into a supernatant and a precipitate, and the precipitate was washed with the MES buffer. 0.5 mg of the anti-CRP monoclonal antibody (Lot. 050921, 030700, or CP80105) was added thereto and stirred at 37° C. for 30 minutes. After the stirring, the mixture was centrifuged (16000 rpm, 4° C., 20 min) and divided into a supernatant and a precipitate. The supernatant was used in the subsequent operation for quantifying the amount of the anti-CRP antibody bound to the latex particles.

The precipitate was suspended in the MES buffer, and 1 mL of denatured BSA was added thereto and stirred at 25° C. for 30 minutes to block an anti-CRP antibody-binding site on the surface of the latex particles. After the blocking, the mixture was suspended in 2.0 mL of a 0.1 M Tris-HCl buffer (pH 8.2), and this suspension was used as an anti-CRP monoclonal antibody-sensitized latex reagent.

### (2) Evaluation of Latex Reagent

The latex reagent prepared in item (1) mentioned above was used in the detection of the rCRP obtained in Example 1. In the assay, LPIA-500 (manufactured by Mitsubishi Kagaku Iatron, Inc.) was used, and the agglutination rate was measured at a wavelength of 800 nm. This measurement was performed with the antigen concentration set to 0 to 100 mg/ml. CRP quantification and detection limit determination were performed based on a calibration curve prepared from the average reaction rate of the latex reagent.

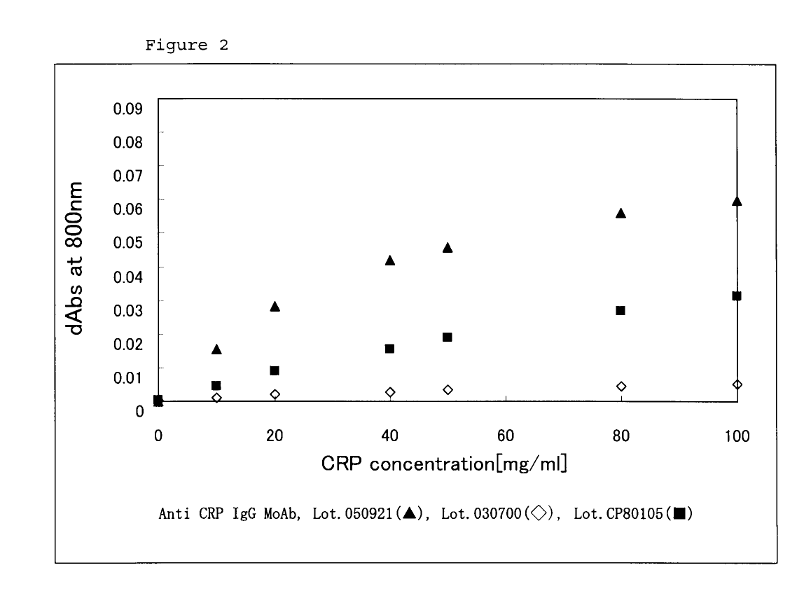

As a result, the Lot. 050921-sensitized latex reagent exhibited highly sensitive and stable measurement values of 3 ng/ml to 0.0596 mg/ml as shown in FIG. 2. Moreover, the Lot. CP80105-sensitized latex reagent exhibited 0.0002 to 0.0313 mg/ml, and the Lot. 030700-sensitized latex reagent exhibited 0.0002 to 0.005 mg/ml.

### Test Example 2

### CRP Assay in Human Serum

The latex reagent (Lot. 050921 or CP80105) prepared above was used to assay a CRP concentration in 30 mL of serum obtained by blood collection from patients with liver disease. The results of assay using each anti-CRP monoclonal antibody-sensitized latex reagent were compared with results of assay using an anti-CRP polyclonal antibody-sensitized latex reagent (anti-CRP PoAb-sensitized latex reagent).

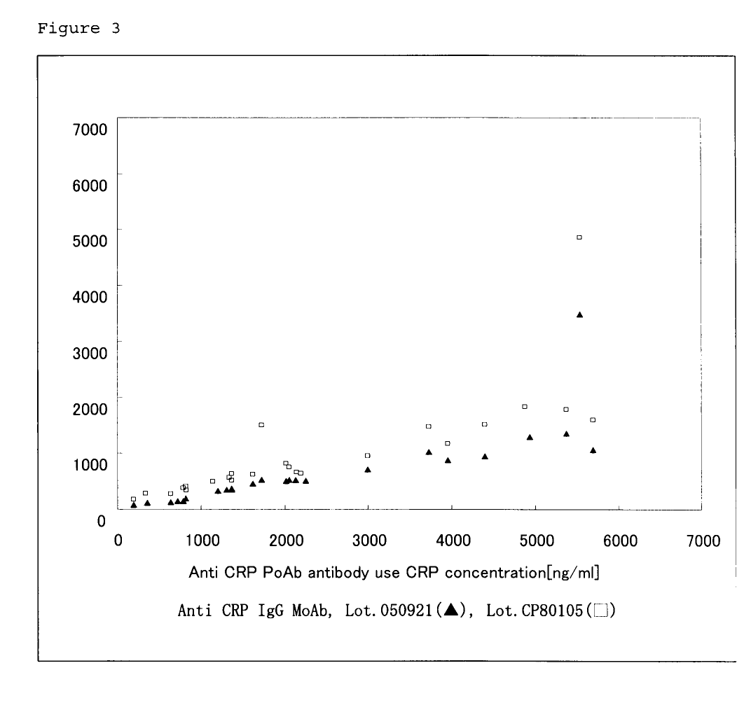

As a result, the measured value in the liver disease specimen in the assay using the anti-CRP monoclonal antibody Lot. 050921-sensitized latex reagent was about half that obtained using the anti-CRP PoAb-sensitized latex reagent as shown in FIG. 3. This is probably because the anti-CRP PoAb-sensitized latex reagent has a large number of epitopes which are likely to exhibit nonspecific reaction with other substances, whereas the antibody of the present invention exhibits specific reaction only with one epitope. Moreover, high reactivity is also shown in comparison with the anti-CRP monoclonal antibody Lot. CP80105-sensitized latex reagent.

1. An anti-CRP antibody which reacts with a C-reactive protein (hereinafter referred to as CRP) and recognizes an epitope located at residues 147 to 172 in a CRP amino acid sequence represented by SEQ ID NO: 1.

2. The anti-CRP antibody according to claim 1, wherein the anti-CRP antibody is a monoclonal antibody.

3. The anti-CRP antibody according to claim 2, wherein the anti-CRP antibody is produced by a hybridoma deposited under Accessition No. FERM ABP-11001.

4. The anti-CRP antibody according to any one of claims 1 to 3, wherein the anti-CRP antibody is obtained using a peptide having the amino acid sequence represented by SEQ ID NO: 1 as an immunogen.

5. The anti-CRP antibody according to claim 4, wherein the peptide is a recombinant peptide.

6. A hybridoma CRP8 (Accession No. FERM ABP-11001).

7. A CRP assay reagent comprising the anti-CRP antibody according to any one of claims 1 to 5.

8. A method of CRP assay comprising bringing an anti-CRP antibody according to any one of claims 1 to 5 into contact with a test sample, and conducting an immunoassay.

9. The method according to claim 8, wherein the test sample is blood, serum, or plasma.

10. The method according to claim 8 or 9, wherein means for the immunoassay is ELISA or a latex agglutination method.


SEQUENCE LISTING (9 sequences)

SEQ ID NO: 1 - protein, 206 aa, Homo sapiens
  CRP
QTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 2 - DNA, 28 bp, Artificial Sequence
  Description of Artificial Sequence Synthetic primer for amplification of human CRP
cacccagacagacatgtcgaggaaggtt

SEQ ID NO: 3 - DNA, 23 bp, Artificial Sequence
  Description of Artificial Sequence Synthetic primer for amplification of human CRP
caccatgtcgaggaaggcttttg

SEQ ID NO: 4 - DNA, 24 bp, Artificial Sequence
  Description of Artificial Sequence Synthetic primer for amplification of human CRP
cacctcgtatgccaccaagagaca

SEQ ID NO: 5 - DNA, 25 bp, Artificial Sequence
  Description of Artificial Sequence Synthetic primer for amplification of human CRP
caccagggtgaggaagagtctgaag

SEQ ID NO: 6 - DNA, 21 bp, Artificial Sequence
  Description of Artificial Sequence Synthetic primer for amplification of human CRP
caccgaaggaagccagtccct

SEQ ID NO: 7 - DNA, 22 bp, Artificial Sequence
  Description of Artificial Sequence Synthetic primer for amplification of human CRP
caccaccatctatcttggcggg

SEQ ID NO: 8 - DNA, 21 bp, Artificial Sequence
  Description of Artificial Sequence Synthetic primer for amplification of human CRP
aaaccccagctgtggccctga

SEQ ID NO: 9 - DNA, 1182 bp, Homo sapiens
tttgcttcccctcttcccgaagctctgacacctgccccaacaagcaatgttggaaaattatttacatagtggcgcaaactcccttactgctttggatataaatccaggcaggaggaggtagctctaaggcaagagatctgggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacaggtaagggccaccccaggctatgggagagttttgatctgaggtatgggggtggggtctaagactgcatgaacagtctcaaaaaaaaaaaaaaaagactgtatgaacagaacagtggagcatccttcatggtgtgtgtgtgtgtgtgtgtgtgtgtgtgtggtgtgtaactggagaaggggtcagtctgtttctcaatcttaaattctatacgtaagtgaggggatagatctgtgtgatctgagaaacctctcacatttgcttgtttttctggctcacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctga